(19) United States
(12) Patent Application Publication
(10) Pub. No.: US 2016/0318006 A1
(43) Pub. Date: Nov. 3, 2016

(54) Low Pressure Dimethyl Ether Synthesis Catalyst

(71) Applicant: COOL PLANT ENERGY SYSTEMS, INC., Greenwood Village, CO (US)

(72) Inventors: Rajashekharam MALYALA, Camarillo, CA (US); Vern S. TRAXLER, Simi Valley, CA (US); Mark L. JARAND, Newbury Park, CA (US); Timothy Alan THOMPSON, Ventura, CA (US); Michael C. CHEIKY, Thousand Oaks, CA (US)

(73) Assignee: Cool Planet Energy Systems, Inc., Greenwood Village, CO (US)

(21) Appl. No.: 15/104,708

(22) PCT Filed: Dec. 16, 2014
(86) PCT No.: PCT/US14/70618

§ 371 Date: Jun. 15, 2016

Related U.S. Application Data
(60) Provisional application No. 61/916,389, filed on Dec. 16, 2013.

(51) Int. Cl. B01J 29/40; B01J 21/04; B01J 23/80; B01J 23/889; C07C 41/09; C07C 29/153

(52) U.S. Cl. CPC B01J 29/40; C07C 41/09; C07C 29/153; B01J 23/80; B01J 23/8892; B01J 21/04

(57) ABSTRACT

A catalyst and process for synthesis of dimethyl ether from synthesis gas are disclosed. The catalyst and process allow dimethyl ether synthesis at low pressures (below 20 bars) at a conversion rate close to the expected equilibrium rate. The catalyst is a combination of a methanol synthesis catalyst with metal components comprising Cu, Zn, Al, Mn and Cs and a methanol dehydration catalyst, wherein the dehydration catalyst is a dehydration agent which allows optimum production of dimethyl ether.

### REFERENCE TO RELATED APPLICATIONS

The application claims the benefit of priority under 35 U.S.C. §119(e) to U.S. Provisional Application Ser. No. 61/916,389 filed Dec. 16, 2013, the contents of which is incorporated herein by reference in its entirety.

This application is related to U.S. Ser. No. 13/567,991, filed Aug. 6, 2012, and entitled “Low Pressure Dimethyl Ether Synthesis Catalyst”, which claims priority to U.S. Provisional Patent Application No. 61/530,813, filed on Sep. 2, 2011, the contents of which is incorporated herein by reference in their entirety.

### TECHNICAL FIELD

The present invention relates generally to catalysis, and more particularly to a dimethyl ether synthesis catalyst that operates efficiently at low pressures.

### DESCRIPTION OF THE RELATED ART

Dimethyl ether is a versatile compound capable of being used as a combustion fuel, a cooking fuel, an additive to liquefied propane gas, and an intermediate for the production of other chemical compounds. The basic steps in the dimethyl ether synthesis from synthesis gas are as are as follows:

CO+2H₂→CH₃OH 1)

2CH₃OH→CH₃OCH₃+H₂O 2)

Equilibrium syngas conversion is increased as the methanol formed undergoes dehydration to generate dimethyl ether (DME). The water gas shift reaction (WGS) is also involved as a side reaction leading to the formation of carbon dioxide and hydrogen according to the following equation:

CO+H₂O→CO₂+H₂ 3)

When all 3 reactions happen in a single reactor the process is known as direct conversion of syngas to DME (STD). In this case the net reaction is:

3CO+3H₂→CH₃OCH₃+CO₂ 4)

The DME catalyst is a combination of methanol synthesis catalyst and dehydration catalyst. The ratio of methanol synthesis catalyst to dehydration catalyst is typically less than 3:1. In other words the amount of dehydration component in the catalyst is typically more than 25%, although lower dehydration catalysts loads have been reported. Acidic materials such as alumina, silica, silica-alumina and zeolites have been used in the DME catalyst as dehydrating components. Zeolites such as ZSM-5, zeolite Y and SAPO have been used as dehydrating agents in the DME catalyst formulation. These zeolites are expensive. Therefore, it is desirable to have a DME catalyst that has the minimal amount of dehydration catalyst in the formulation, especially when zeolites are used as the dehydrating agents and still be able to produce the stoichiometric amount of DME as described by the above overall equation.

Additionally, the catalyst is in pellet form. Addition of dehydrating agent such as zeolites to the catalyst, while needed, reduces the mechanical integrity of the catalyst. It also reduces the pellet density and compact bulk density of the final catalyst. This is because the dehydrating agent is more porous with excess surface area when compared to the methanol synthesis catalyst. Having a minimal amount of dehydrating agent while still meeting the desired DME output is thus advantageous, not only from a catalyst cost perspective, but also in terms of catalyst mechanical stability and from a methanol synthesis component dilution stand point. A catalyst with lower compact bulk density and lower pellet density might need a physically larger reactor to convert the synthesis gas to DME, thus increasing the overall capital expenditure in a commercial plant.

The rate determining step in the dimethyl ether synthesis process is believed to be the methanol synthesis reaction. Intensive efforts have been made to find suitable catalysts which operate under mild conditions. The original catalysts for methanol synthesis were comprised of ZnO and of ZnO/Cr₂O₃. These catalysts allowed synthesis pressures of 300 to 400 bar and synthesis temperatures of 350° C. starting from synthesis gas. Subsequent work by ICI Corp. led to the development of copper based catalysts, of the form Cu/ZnO/Al₂O₃ and Cu/ZnO/Cr₂O₃, termed low pressure catalysts, which allowed commercial operation at synthesis pressures of 30-90 bars and synthesis temperatures of 220° C. to 300° C. Such a methanol synthesis catalyst coupled with alumina or a zeolite such as ZSM-5 is typically used as a DME catalyst, in the direct conversion of synthesis gas to DME. One such commercial catalyst, for example, is disclosed in U.S. Pat. No. 7,033,972, assigned to JFE Holdings. The catalyst includes a methanol synthesis catalyst formed around small sized (200 microns or less) alumina particles. Reaction pressure using this catalyst is typically 50 bars. The capital costs to achieve production on largest scale even with these “low” pressures can be considerable. Catalysts capable of DME production at lower pressures can significantly lower both capital costs of installation design and continuous operation costs.

The use of dehydration components along with the methanol synthesis catalyst in current DME catalysts increases the overall cost of the catalyst and the reactor to produce DME from synthesis gas. Furthermore, the catalyst operate at pressures greater than 30 bars and in many cases greater than 50 bars to produce effective DME from synthesis gas. Having a catalyst producing the required amounts of DME at lower pressures is attractive as it reduces the capital expenditure significantly.

### SUMMARY

DME catalysts with high efficiency conversion of synthesis gas into dimethyl ether at low pressures (e.g., below 20 bar) are disclosed. The heterogeneous catalysts for the conversion of syngas to dimethyl ether demonstrate high efficiency (e.g., greater than 60% conversion) at pressures lower than 20 bar.

In one aspect, a catalyst composition for the synthesis of dimethyl ether from synthesis gas, includes (a) a methanol synthesis component comprising copper, zinc, aluminum and manganese oxides, wherein an atomic ratio of Zn to Cu is 0.05 to 2; wherein an atomic ratio of Al to Zn is 0.1 to 10; and wherein manganese oxide content is less than 10 wt %; and (b) a dehydration component comprising an acid catalyst having one or more solid acidic components; wherein the weight ratio of methanol synthesis component to dehydration component is between 4:1 to 20:1.

In one or more embodiments, the weight ratio of methanol synthesis component to dehydration component is between 5:1 to 10:1, or the weight ratio of methanol synthesis catalyst component to acidic dehydration catalyst greater than 4 to 1, or greater than 5 to 1, or greater than 6 to 1, or greater than 7 to 1, or greater than 8 to 1, or greater than 9 to 1 or greater than 10 to 1, or the DME catalysts includes about 5 wt % to about 25 wt %, or about 10 wt % to about 20 wt %, of an acidic dehydration catalyst

In any of the preceding embodiments, the methanol synthesis component further comprises cesium and/or the cesium content is less than 1.0 wt %, or the cesium content is in the range of 0.001-1.0 wt %, or the cesium content is in the range of 0.05-0.5 wt %.

In any of the preceding embodiments, the manganese oxide content is in the range of 0.5-10 wt %.

In any of the preceding embodiments, the dehydration component is selected from at least one of the group consisting of: silica alumina, kaolin, gamma alumina, aluminum silicate, montmorillonite, mullite, mesostructured aluminosilicate, and zeolites and/or the dehydration component is selected from zeolite-Y, ZSM-5 and SAPO.

In any of the preceding embodiments, the dehydration component is separately calcined from the methanol synthesis component, and/or for example, the dehydrating component is calcined at temperatures exceeding 500° C., and/or for example, the methanol synthesis component is calcined at temperatures below 400° C.

In any of the preceding embodiments, the dehydration component is produced using pore former materials selected from the group consisting of: microcrystalline cellulose, starch, lignocellulosic compounds, acrylates, carboxylates, and sulfonates.

In any of the preceding embodiments, the dehydration agents cause a temperature rise of between 0.8° C. and 2° C. when 2.000 g of the dehydrating agents is calorimetrically titrated against a 20% butylamine/hexane solution.

In any of the preceding embodiments, the catalyst composition is in the form of a pellet, and/or, for example, the pellet density is 2-3 g/cc, and/or for example the compact bulk density of the pellet is about 1-2 g/cc and more preferably about 1.7 g/cc.

In another aspect, a method of producing dimethyl ether includes contacting synthesis gas comprising hydrogen and carbon monoxide with a catalyst as described herein.

In one or more embodiments, the synthesis gas further includes carbon dioxide, and/or the synthesis gas further comprises of methane.

In any of the preceding embodiments, the reaction pressure is 20 bar or less.

In any of the preceding embodiments, the conversion rates of synthesis gas into dimethyl ether is greater than 60%.

As the density of this catalyst is much higher than the density of a catalyst having 25% or more dehydrating agent, it is possible to reduce the overall reactor size. This is important as it results in less in capital expenditure for industrial installations.

### BRIEF DESCRIPTION OF THE DRAWING

The present invention, in accordance with one or more embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

FIG. 1 is a chart including graphs of calculated equilibrium carbon monoxide conversion to dimethyl ether versus reactor pressure for different temperatures.

FIG. 2 is a schematic illustrating the equipment used in the synthesis of dimethyl ether from synthesis gas.

FIG. 3 shows the reduction profile of the catalyst as recorded by the residual gas analyzer.

FIG. 4 show gas chromatograms of input and output samples from which CO conversion and % DME in the composition was evaluated.

FIG. 5 shows the RGA data obtained for extended periods of time using the catalyst showing DME production and CO conversion.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION

The present invention is directed toward a heterogeneous catalyst which allows efficient syngas conversion to dimethyl ether at pressures lower than those used in present commercial systems. This catalyst comprises a mixture of a methanol synthesis catalyst and an acidic dehydration catalyst, in which the weight ratio of methanol synthesis catalyst component to acidic dehydration catalyst ratio by weight is greater than 3 to 1, or greater than 4 to 1, or greater than 5 to 1, or greater than 6 to 1, or greater than 7 to 1, or greater than 8 to 1, or greater than 9 to 1 or greater than 10 to 1. In other embodiments, the methanol synthesis catalyst component to acidic dehydration catalyst ratio by weight is in the range of 3:1 to 10:1, or the methanol synthesis catalyst component to acidic dehydration catalyst ratio by weight is in the range of 4:1 to 9:1, or is a range bounded by any value disclosed hereinabove. In other words, the amount of acidic dehydration component in the catalyst is below 33 wt %. In one or more embodiments, the DME catalysts includes about 5 wt % to about 25 wt %, or about 10 wt % to about 20 wt %, of an acidic dehydration catalyst. The DME catalyst includes a methanol synthesis catalyst that is especially well suited for operation at low pressures.

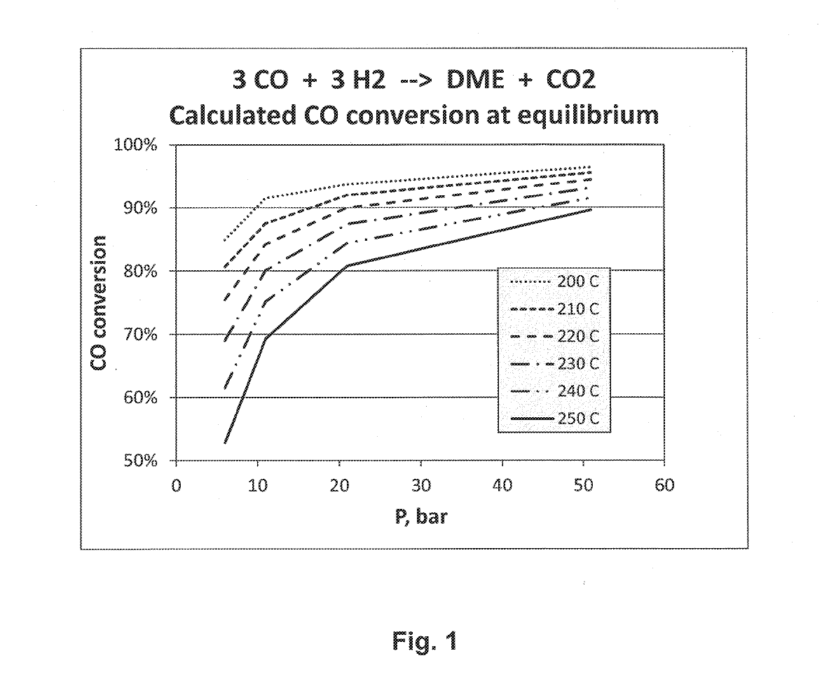

The difficulty of operating at low pressures is evident from an examination of FIG. 1, which shows calculated equilibrium curves for the conversion of synthesis gas to dimethyl ether as a function of pressure for different temperatures. The conversion rates are shown for temperatures from 200° C. to 250° C. The conversion rates start to decline significantly at pressures below 20 bar. Present commercial catalysts are optimized to work above 30 bar. A catalyst that may be suitable at 50 bar may underperform at pressures below 20 bar. The catalyst of the present invention is optimized to operate at the lower pressures. In some embodiments, the catalyst is optimized to operate at below 20 bar, and for example, at 10 bar and below. In some embodiments, catalyst can operate within a range of 29 bar to 5 bar, or 20 bar to 10 bar.

A bi-functional catalyst for conversion of synthesis gas to dimethyl ether is described. The catalyst includes two types of active sites—methanol formation sites and methanol dehydration catalytic sites. The methanol synthesis catalyst component to acidic dehydration catalyst ratio by weight is greater than 3 to 1, or greater than 4 to 1, or greater than 5 to 1, or greater than 6 to 1, or greater than 7 to 1, or greater than 8 to 1, or greater than 9 to 1 or greater than 10 to 1. In other embodiments, the methanol synthesis catalyst component to acidic dehydration catalyst ratio by weight is in the range of 3:1 to 10:1, or the methanol synthesis catalyst component to acidic dehydration catalyst ratio by weight is in the range of 4:1 to 9:1, or is a range bounded by any value disclosed hereinabove. In other embodiments, the amount of acidic dehydration component in the catalyst is below 33 wt %. In one or more embodiments, the DME catalysts includes about 5 wt % to about 25 wt %, or about 10 wt % to about 20 wt %, of an acidic dehydration catalyst.

The reaction chemistry of DME formation is such that synthesis gas reacts on the catalyst to first form methanol. This methanol is dehydrated to form DME. As the equilibrium is shifted towards the right hand side, the dehydrating catalyst allows the methanol synthesis catalyst to produce more methanol. Thus a combination of highly active methanol synthesis catalyst with a highly active dehydrating catalyst may result, favorably producing close to equilibrium amounts of DME from synthesis gas. Zeolites are examples of materials with high dehydrating activity, generated from their acidity characteristics. They also have very high surface area and significant porosity.

In one aspect, the bi-functional catalyst includes a methanol formation catalyst that is selected for efficient operation at low pressures, e.g., below 20 bar, and in preferred embodiments, at 10 bar or at 5 bar and below, or 5-20 bar or 10-20 bar. According to one or more embodiments, the methanol synthesis catalyst includes co-precipitated oxides of copper, zinc, aluminum and manganese, e.g., CuO/ZnO/Al₂O₃/MnO catalyst. Typical metal ratios of Cu to Zn may vary from 5:1 to 1:5. In the case of an aluminum oxide, Al to Cu metal ratio may vary from 0.05 to 2 and Al to Zn metal ratio may vary from 0.1 to 10, or 0.1 to 1. Manganese oxide can be present at less than 10 wt % of the total methanol synthesis catalyst. In some embodiments, manganese oxide content is about 0.05-10 wt %, or preferably about 0.05-5 wt %. The catalyst may optionally contain cesium. Cesium can be present at less than 1 wt % of the total of the total methanol synthesis catalyst. In some embodiments, cesium content is about 0.001-1.0 wt %, or preferably about 0.05-0.5 wt %.

Tan et al (Catalysis Today 104 (2005) 25-29) have shown that a Mn doped catalyst is useful for DME synthesis during liquid phase synthesis of DME from syngas. They performed their reactions at 50 to 100 atmospheres pressure, using a catalyst containing 33% dehydration component, e.g., a 2:1 ratio of methanol synthesis to methanol dehydration catalyst, and reported 50 to 70% CO conversion depending on operating conditions. Fie et al (Energy Fuels, 2004, 18 (5), pp 1584-1587) documented the effect of Mn and Zn addition on copper based DME catalysts containing zeolite Y. They report that the conversion of CO and the selectivity of DME (e.g., the formation of DME over other alternative reaction products) is 53.6 mol % and 63.5 mol % on Cu—Mn—Zn/zeolite-Y, respectively. Zin et al (Journal of Molecular Catalysis A: ChemicalVolume 176, Issues 1-2, 20 Nov. 2001, Pages 195-203) speculated that the addition of Mn increases the dispersion of copper in the DME catalysts and this could be responsible for the higher activity of copper based methanol/DME synthesis catalysts. Much of these operations were performed at pressures greater than 20 bars, using powder based catalysts, far from commercial catalysts that use pellet based formulations.

In one aspect of the invention, the methanol synthesis catalyst composition is selected to reduce the amount of dehydration component of the DME catalyst without negatively affecting conversion of CO and the selectivity of DME. It was found that the addition of manganese and optionally cesium to the methanol formation catalyst not only improved the efficiency of the syngas conversion, but also reduced the amount of zeolite catalyst required as a dehydration component. This permits low operating pressures while maintaining conversion rates of synthesis gas of greater than 60%.

The manganese addition to the methanol synthesis catalyst not only has a positive effect on syngas to methanol conversion, it is also has a beneficial role in the DME catalyst, thereby potentially reducing the overall effective load of methanol dehydration catalyst from other sources (such as zeolites, alumina, clays and other traditional acidic dehydration catalysts) without loss of catalytic function and efficiencies. In one or more embodiments, the DME catalysts includes about 5 wt % to about 25 wt %, or about 10 wt % to about 20 wt %, of an acidic dehydration catalyst.

While the presence of manganese has been found effective in improving performance of the syngas to DME conversion, manganese also has been associated with increases in agglomeration, resulting in larger particle size. Agglomeration reduces surface area and results in uneven particle size distributions that can give uneven and inconsistent catalytic performance. It has been surprisingly determined that addition of cesium to the methanol formation catalyst reduces agglomeration and enhances the performance of the catalyst. Without being bound by any particular mode of operation, it is believed that the cesium can reside in the defect sites in the oxide matrix, allowing the reduction of metal/metal oxide agglomeration. Alternatively, cesium may interact with manganese to stabilize its oxidation state. In some embodiments, manganese oxide content is about 0.05-10 wt %, or preferably about 0.05-5 wt %—The catalyst may optionally contain cesium. Cesium can be present at less than 1 wt % of the total of the total methanol synthesis catalyst. In some embodiments, ceria content is about 0.001-1.0 wt %, or preferably about 0.05-0.5 wt %.

In one or more embodiments, the Mn/Cs ratio can 10 in the catalyst; however, the ratio can be varied and is not limited to this ratio. For example, the Mn/Cs ratio can be 15 or 14, or 13, or 12, or 11, or 10, or 9, or 8, or 7 or can be a range bounded by any of these values.

The catalyst is expected to convert syngas to DME. Methanol is formed as a precursor to DME formation. If more methanol is formed than it is converted to DME, then it is not a good DME catalyst, although it results in decent syngas conversion. Thus in addition to syngas conversion, DME selectivity is important. Typically, if the catalyst is short of acidic component, it undermines DME formation despite good syngas conversion.

The methanol synthesis catalyst can be prepared by co-precipitation of the constituent oxides, from nitrate salts e.g., nitrate salts of copper, zinc, aluminum, manganese and (optionally) cesium. Generally co-precipitation is effected by addition of a basic salt such as sodium carbonate, sodium bicarbonate, ammonium carbonate, ammonium bicarbonate or ammonium hydroxide. In other embodiments, nitrate salts of copper, zinc, aluminum and manganese are co-precipitated, while cesium is blended optionally into the formed carbonates in a separate step. These carbonates may be co-precipitated with various metal oxides and/or metal salts known to those skilled in the art, including oxides of chromium, zirconium and boron. Co-precipitation may also be performed onto a sol or onto a suspension of well dispersed solid particles.

After precipitation, the precipitate is filtered, washed and rinsed to remove salt impurities. The clean precipitate is then dried to remove all water and calcined at temperatures from 250° C. to 400° C. to effect full conversion of any remaining carbonates to its corresponding oxides. The final reduced catalyst is believed to comprise Cu/CuO crystallites well dispersed on oxygen vacancies in a ZnO matrix. Too high a calcination temperature can cause sintering of the precursor Cu/CuO crystallites and reduce catalyst efficiency. After calcination the methanol synthesis powder is further pulverized to attain a suitably large surface area. In some embodiments, the catalyst surface area, as determined via BET method using nitrogen, should preferably exceed 50 m²/g, and most preferably exceed 100 m²/g.

The dehydration catalyst serves the role of dehydrating methanol to DME and further pushing the equilibrium of the synthesis gas conversion towards DME production. In one or more embodiments, solid acids such as silica alumina, gamma alumina, activated alumina or ZSM-5 can be used to effect this dehydration. The dehydration catalyst necessitates high calcination temperatures (>400° C.) for the generation of active acid sites, and the dehydration catalyst should be separately calcined from the methanol synthesis catalyst in order to achieve independent activation of both components. In one or more embodiments, the DME catalysts includes about 5 wt % to about 25 wt %, or about 10 wt % to about 20 wt %, of an acidic dehydration catalyst.

Acidity of the catalyst affects the performance of the dehydration reaction. If the acidity of the dehydration catalyst component is low, the resulting catalyst will exhibit low activity as it cannot convert the methanol formed to DME, thereby affecting the equilibrium synthesis gas conversion. If the acidity of the dehydration compound is high, the resulting catalyst will further dehydrate the DME formed to hydrocarbons, thus affecting the production rate of DME. If the acidity of the dehydration component is too high, then it can cause coking of the feed resulting in a deactivated catalyst affecting DME selectivity. The dehydration component in thus able to control the DME selectivity.

Suitable acid catalysts for the present invention are heterogeneous (or solid) acid catalysts having one or more solid acidic components. Solid acid catalysts that can be combined include, but are not limited to, (1) heterogeneous heteropolyacids (HPAs) and their salts, (2) natural clay minerals, such as those containing alumina, or silica (including zeolites), (3) cation exchange resins, (4) metal oxides, (5) mixed metal oxides, (6) inorganic acids or metal salts derived from these acids such as metal sulfides, metal sulfates, metal sulfonates, metal nitrates, metal phosphates, metal phosphonates, metal molybdates, metal tungstates, metal borates, and (7) combinations of groups 1 to 6.

Suitable HPAs include compounds of the general Formula XₐM_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Methods for preparing HPAs are well known in the art. Natural clay minerals are well known in the art and include, without limitation, kaolinite, bentonite, attapulgite, montmorillonite and zeolites. When present, the metal components of groups 4 to 6 may be selected from elements from Groups I, IIa, IIIa, VIIa, VIIIa, Ib and IIb of the Periodic Table of the Elements, as well as aluminum, chromium, tin, titanium and zirconium. Fluorinated sulfonic acid polymers can also be used as solid acid catalysts for the process of the present invention.

In one or more embodiments of the invention, the dehydration catalyst component is chosen to have an acidity range which optimizes the production of dimethyl ether at pressures below 20 bars, while minimizing the production of methanol and other unwanted hydrocarbons. This acidity range corresponds to acidity values lying in between and including the acidity values of pure gamma alumina and the acidity values of pure calcined ZSM-5. Oxide acidities can be determined for example by tritating the dehydrating agents with 20% N-butylamine/hexane. While it is recognized that the actual acidity of the catalysts in situ in their dehydrated and/or deammoniated forms may be orders of magnitude higher than at ambient conditions, the butyl amine/hexane room temperature calorimetric titration is expected to correlate with the in situ acidities. The following results were observed when 2.000 g of the dehydrating component is treated with 20% N-butylamine/hexane mixture:

| Dehydrating Agent | Temp Rise (° C.) | ml titrated |
|---|---|---|
| γ-alumina | 0.538 | 1.8043 |
| ZSM-5 | 1.690 | 2.2085 |
| Silica Alumina Catalyst | 1.518 | 0.8049 |
| Support | | |
| HZSM-5 + γ-Al₂O₃ | 1.256 | 2.5148 |
| Silica alumina + γ-Al₂O₃ | 1.126 | 1.486 |

The temperature rise is an indication of the strength of the acid sites, while the number of milliliters titrated is an indication of the total number of acid sites. Gamma alumina has weakly acidic sites while ZSM-5 has the strong acidic sites compared to the other formulations.

Dehydrating agent combinations which produce a butylamine titration temperature rise in the range of 0.8° C. to 2° C. are effective dehydrating catalyst components for optimum DME generation for pressures below 20 bar.

In one or more embodiments of the invention, the dehydration catalyst component is comprised of a mixture of one or more of the following dehydration agents: silica alumina, gamma alumina, kaolin, ZSM-5. In one or more embodiments of the invention, the dehydration catalyst component is comprised of a mixture of two or more of the following dehydration agents: 20-40% silica alumina, 10 to 30% gamma alumina, 10-50% kaolin, 25%-75% ZSM-5.

The productivity of DME in such single stage catalytic conversion of synthesis gas not only depends on the relative amounts of methanol synthesis catalyst and the methanol dehydration component, but also on how they are mixed together. The acidity of the zeolite and its availability are both factors in providing a good DME catalyst. Extensive grinding of the two catalyst powders to create a homogeneous mixture may not result in a good DME catalyst as the methanol synthesis catalyst physically blocks the active sites on the zeolite needed for dehydration of the methanol to DME. Thus, it appears that availability of acid sites of dehydration catalyst is a factor for a good DME catalyst. The lack of availability also increases the need to enhance the amount of dehydration component in the final catalyst formulation. Catalysts and methods of producing such catalysts described in this present invention have solved such problems resulting in highly active DME catalyst formulations that can operate at pressures below 20 bars and have dehydration catalyst component below 25%.

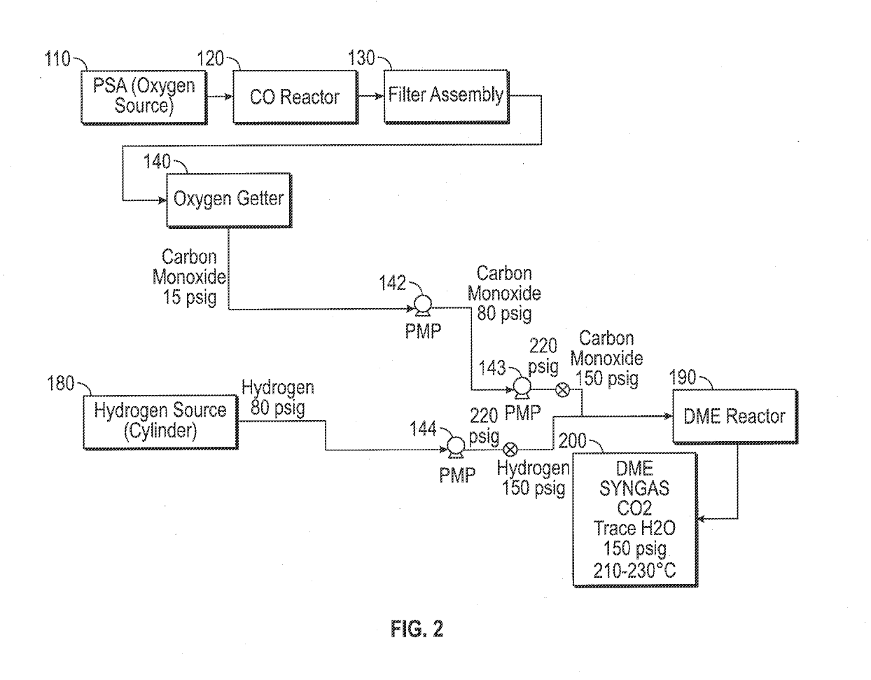

An exemplary experimental set-up for use in converting synthesis gas to dimethyl ether is shown in FIG. 2. The figure shows a schematic of the experimental setup to determine conversion rates from synthesis gas to DME. Carbon Monoxide is generated from reaction of oxygen (after a pressure swing adsorption process 110) with biochar in reactor 120 and passed through filter assembly 130 and oxygen getter 140. The generated carbon monoxide passes through a first pump 142, which compresses it to approximately 80 psig for example and then to a secondary pump 143, which performs a second compression, for example, to 220 psig. Hydrogen is introduced from a cylinder at 40 psig for example and compressed via pump 144 to 220 psig for example. Both gases are metered through needle valves into a mixing and preheating chamber, and finally into the catalyst chamber at 150 psig (ca 10 bar). The reactor temperature is varied between 200° C. and 270° C. at a flow rate space velocity corresponding to 640 hr⁻¹.

The following examples are presented for the purpose of illustration only and are not intended to be limiting of the invention, the full scope of which is set forth in the claims that follow.

### EXAMPLE 1

The two components of the dimethyl ether synthesis catalyst were made as follows:

The CuO/ZnO/Al₂O₃ methanol synthesis catalyst was prepared by a conventional co-precipitation method. HZSM-5 zeolite acid function was prepared by calcining a commercial NH₄- ZSM-5 zeolite. ZSM-5 powder was blenderized and calcined at 550° C. in a static air furnace for 8 hours. The bi-functional DME catalyst was prepared by physically mixing the dry metallic function and the acid function powders at desired ratios and mechanically pelletizing them using a suitable lubricant (2% graphite).

The methanol synthesis catalyst has dopants such as Mn and Cs which are not typical of a commercial methanol synthesis catalyst and the resultant DME catalyst as well. In one embodiment, the catalyst is prepared by metal nitrates co-precipitation at a metal ratio of Cu/Zn/Mn/Cs/Al at 60/30/5.0/0.25/4.4. The following nitrate precursors were weighed and placed in 1.5 L metal container.

Cu(NO₃)₂×2.5H₂O-180.0 g

Zn(NO₃)₂×6H₂O-115.25 g

Al(NO₃)₃×9H₂O-18.3 g

Mn(NO₃)₂×4H₂O-16.5 g

1.3 L De-ionized water was added to the metal nitrate powders. This mixed nitrate solution in water was heated using a hot plate at 80° C. while stirring. Separately, 300 grams of NaHCO₃ is dissolved in 3 liters of de-ionized water and heated in another hot plate to 80° C. while stirring. A 2 gallon stock pot with 1.5 liters of de-ionized water is set-up. This is stirred slowly not to cause excessive splash. A pH meter and thermocouple are used to monitor pH and reaction temperature in the 2 gallon stock pot. This pot is fitted with additional funnels to accept hot liquid additives with controlled addition. The temperature of the contents of this stock pot is 80° C. with stirring. The nitrate solution and the NaHCO₃ solution are added into the funnels separately and their rate of drop wise addition into the 2 gallon stock pot is adjusted so that the pH is at 7±0.1. The rate of drop wise addition of both solutions simultaneously is expected to take place over a period of 45-60 minutes. The metal nitrates react with the NaHCO₃ to precipitate as corresponding carbonates. When the addition is complete, stirring is turned off to allow the carbonate precipitate to settle for 5-10 minutes. The liquid is decanted and the precipitate is collected in a separate container. This container is placed in a convection oven at 80° C. for 2 hours to age the precipitate. Upon completion of these 2 hours, the slurry is aged. The precipitate is vacuum filtered, and washed with 4 liters of de-ionized water at 80° C. The filtered cake (mostly in thick viscous slurry form) is then transferred into a stainless steel pan. 0.64 grams of cesium nitrate is added to the filtered cake and mixed thoroughly. The cake is then subject to drying at 110° C. for overnight.

Calcination of the Precipitate from Carbonate to Oxide

The dried chunks of material is collected and pulverized using a blender. Calcination is performed on this powder at 350° C. for 8 hours in a static air furnace.

### Press Pellet Production

Dimethyl ether (DME) Catalyst comprises typically 2 powders blended together plus a lubricant (typically lubricant is Aldrich 7-11 micron synthetic graphite powder used at 2% total weight to above powder mix).
a. Methanol synthesis powder—as described above
b. Dehydration agent—standard is H form ZSM-5 powder of 23/1 Si/Al ratio
c. Mix methanol synthesis powder/dehydration agent (calcined ZSM5 powder) at 10/1 ratio.

The ratio of methanol synthesis catalyst and HZSM-5 (acid component) are mixed thoroughly in 10:1 ratio. To this 2% graphite is added and mixed well using a high speed blender. The mixture is then roller pressed several times (typically 3 to 4 times) to densify or agglomerate the powder and make it more flaky, granulated, and compact for pellet pressing. The roller pressed powder flakes are run through a 10 mesh screen to lower the size of the particles in order to aid the process of pellet pressing.

### Pelletizing the Powder Mix:

Commercial pelletizer with trade name “Cap Plus” was used to make pressed pellets. The machine had custom made press dies to generate short cylindrical pellets of around 5-6 mm in diameter and 2-3 mm length. The machine is vacuum cleaned and lubricated with mineral oil prior to use. The mixed, roller pressed & screened powdered material is now added into the feed hopper of the pelletizer. Prior to making appropriate pellets, the machine is adjusted accurately by making a few trials of pellets and checking their densities. By adjusting the pressure on the upper die's, the depth of the lower die's in the press cups, pellets of the desired density are produced. Pellets are then screened for any broken or unwanted powders. Pellets are verified for individual pellet density and compact bulk density as well. Pellet samples are further characterized and used in activity evaluation. BET characterization of the pellets revealed a surface area of about 100 m²/g. The pellet density was 2-3 g/cc and more preferably about 2.5-2.7 g/cc. The compact bulk density of the pellets was about 1-2 g/cc and more preferably about 1.7 g/cc.

### EXAMPLE 2

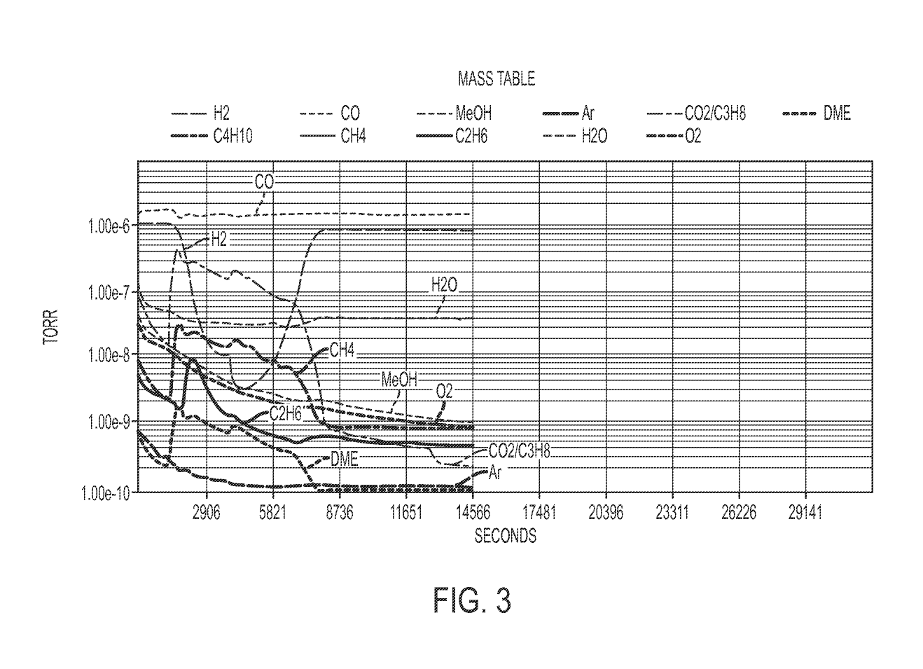

30 grams of catalyst prepared according to the procedure described above, was used in a laboratory DME reactor. The catalyst was reduced using a flow of hydrogen and nitrogen. The ratio of hydrogen to nitrogen used was 1:9 for catalyst reduction. The catalyst reduction was carried at 230° C. initially and at 270° C. for the last one hour. The effluent of the DME reactor was monitored by using a residual gas analyzer (RGA). FIG. 3 shows the reduction profile of the catalyst as recorded by the residual gas analyzer.

For measuring the activity of the catalyst, synthesis gas was obtained as follows: While hydrogen was introduced and compressed from the tank to the reactor, CO was generated by reacting a bed of activated carbon at 910° C. using oxygen separated from air and then compressed into the reactor. The effluent from the CO generator at 910° C. also indicates the presence of equilibrium amounts of CO₂ and CH₄. The gas composition at the entrance of the DME reactor indicated a ratio of H₂:CO:CO₂=10:9:1 approximately. The catalyst was evaluated at approximately 640 hr⁻¹ space velocity at 190-270° C. At 210° C. and 150 psi (ca. 10 bar), the observed CO conversion was around 60% CO with 13.5% DME generation in the gas sample collected after the DME reactor at ambient temperature and pressure.

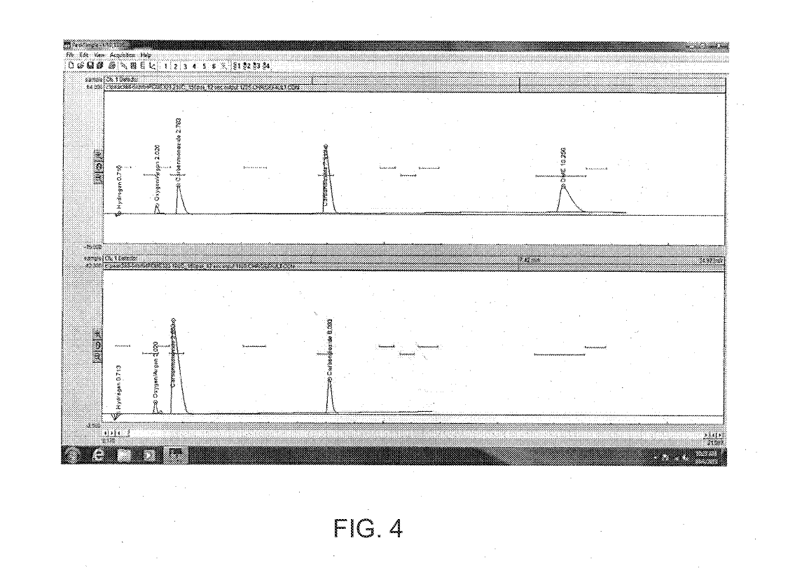

FIG. 4 show gas chromatograms of input (bottom) and output (top) samples from which CO conversion and % DME in the composition was evaluated. Argon was used as the internal standard for calibration purposes.

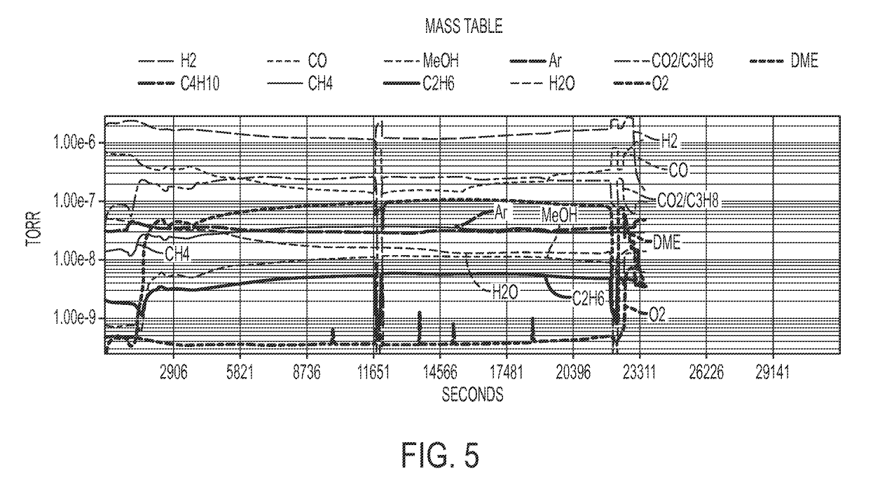

FIG. 5 shows the RGA data obtained for extended periods of time using the catalyst showing DME production and CO conversion. Methanol and water were formed as side products as can be seen from the RGA data. As can be seen from FIG. 5 the amount of methanol and water formed are significantly lower than DME and CO₂. Thus, DME and CO₂ are the major products, following the overall reaction equation represented in (4).

Modifications may be made by those skilled in the art without affecting the scope of the invention. Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. These illustrations and their accompanying description should not be construed as mandating a particular architecture or configuration.

1. A catalyst composition for the synthesis of dimethyl ether from synthesis gas, comprising:
(a) a methanol synthesis component comprising copper, zinc, aluminum and manganese oxides,
wherein an atomic ratio of Zn to Cu is 0.05 to 2;
wherein an atomic ratio of Al to Zn is 0.1 to 10; and
wherein manganese oxide content is less than 10 wt %; and
(b) a dehydration component comprising an acid catalyst having one or more solid acidic components;
wherein the weight ratio of methanol synthesis component to dehydration component is between 4:1 to 20:1.

2. The catalyst composition according to claim 1, wherein the weight ratio of methanol synthesis component to dehydration component is between 5:1 to 10:1.

3. The catalyst composition according to claim 1, wherein the weight ratio of methanol synthesis catalyst component to acidic dehydration catalyst is greater than 4 to 1.

4. The catalyst composition according to claim 1, wherein the DME catalysts includes about 5 wt % to about 25 wt % of an acidic dehydration catalyst.

5. The catalyst composition according to claim 1, wherein the methanol synthesis component further comprises cesium.

6. The catalyst composition according to claim 5, wherein the cesium content is less than 1.0 wt %.

7. The catalyst composition according to claim 5, wherein the cesium content is in the range of 0.001-1.0 wt %.

8. The catalyst composition according to claim 5, wherein the cesium content is in the range of 0.05-0.5 wt %.

9. The catalyst composition according to claim 1, wherein the manganese oxide content is in the range of 0.5-10 wt %.

10. The catalyst composition according to claim 1, wherein the dehydration component is selected from at least one of the group consisting of: silica alumina, kaolin, gamma alumina, aluminum silicate, montmorillonite, mullite, mesostructured aluminosilicate, and zeolites.

11. The catalyst composition according to claim 10, wherein the dehydration component is selected from zeolite-Y, ZSM-5 and SAPO.

12. The catalyst composition of claim 1, wherein the dehydration component is separately calcined from the methanol synthesis component.

13. The catalyst composition according to claim 12, wherein the dehydrating component is calcined at temperatures exceeding 500° C.

14. The catalyst composition according to claim 12, wherein the methanol synthesis component is calcined at temperatures below 400° C.

15. The catalyst composition according to claim 1, wherein the dehydration component is produced using pore former materials selected from the group consisting of: microcrystalline cellulose, starch, lignocellulosic compounds, acrylates, carboxylates, and sulfonates.

16. The catalyst composition according to claim 1, wherein the dehydration agents cause a temperature rise of between 0.8° C. and 2° C. when 2.000 g of the dehydrating agents is calorimetrically titrated against a 20% butylamine/hexane solution.

17. The catalyst composition of claim 1, wherein the composition is in the form of a pellet.

18. The catalyst of claim 17, wherein pellet density is 2-3 g/cc.

19. The catalyst of claim 17, wherein compact bulk density of the pellet is about 1-2 g/cc and more preferably about 1.7 g/cc.

20. A method of producing dimethyl ether, the method comprising:
contacting synthesis gas comprising hydrogen and carbon monoxide with a catalyst according to claim 1.

21. The method of claim 20, wherein the synthesis gas further comprises carbon dioxide.

22. The method of claim 20, wherein the synthesis gas further comprises of methane.

23. The method of claim 20, wherein the reaction pressure is 20 bar or less.

24. The method of claim 20, wherein the conversion rates of synthesis gas into dimethyl ether is 60% or greater.
